(19) United States
(12) Patent Application Publication
(10) Pub. No.: US 2004/0152994 A1
(43) Pub. Date: Aug. 5, 2004

(54) Liver function test

(76) Inventor: Wolfram Meier-Augenstein, Belfast (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/472,431

(22) PCT Filed: Mar. 20, 2002
(86) PCT No.: PCT/GB02/01310

(30) Foreign Application Priority Data
Mar. 20, 2001 (GB) 0106923.6

(51) Int. Cl. A61B 5/08

(52) U.S. Cl. 600/532

(57) ABSTRACT

The present invention relates to a method for testing liver function by for example measuring levels of labelled CO₂? [symbol] in exhaled breath after providing a patient with doses of a labelled substrate capable of being metabolized by the liver to CO₂, a substrate incapable of being metabolised by the liver CO₂. The present invention also provides a kit based on the abovementioned method for testing liver function in a subjects. The method and kit of the present invention can be further applied to the non-invasive monitoring of disease status of a subject on a regular basis and to the detection of changes in liver function of a subject due to adverse drug reactions or chronic medication.

The present invention relates to methods and kits for testing liver function by for example measuring levels of labelled CO₂ in exhaled breath after providing a patient with doses of a labelled substrate capable of being metabolized by the liver to CO₂, and a substrate incapable of being metabolised by the liver to CO₂.

### BACKGROUND

The liver is a major site of intermediary metabolism and central to many processes of fatty acid, amino acid and carbohydrate breakdown and synthesis. Furthermore, the liver is pivotal in bio-transformation of drug precursors as well as clearance and detoxification of drugs and other non-drug xenobiotics such as alcohol and organic solvents. Since the liver possesses substantial reserve capacity in terms of overall function, patients suffering from impaired liver function characteristically present late with clinical symptoms. This fact is one reason for the need of a sensitive liver function test with high selectivity (i.e. no false-negatives) and equally high specificity (i.e. no false-positives).

The other reason is the fact that currently a variety of blood based tests are used such as liver enzyme concentration in the blood stream, to determine liver function. However, it is increasingly apparent that these blood factors, do not necessarily reflect liver function but the integrity of the liver cell (hepatocyte) membrane. Even a tissue sample from a liver biopsy light give a wrong picture if a still healthy part of the liver has been sampled. In addition, these methods are invasive, uncomfortable, and their associated risks make them unsuitable for repeat studies to monitor liver status, especially in children. A breath test, exploiting a metabolic pathway specific to a liver compartment (for instance liver cytosol, liver microsomes or liver mitochondria) may provide an answer to any/all of these problems.

Attempts towards this approach have focused on the non-invasive assessment of nono-oxygenase (cytochrome P450) activity using the ¹³CO₂-breath test principle. This enzyme system is located in the smooth endoplasmatic reticulum (smooth ER) of the hepatocytes and is part of the liver's detoxification machinery. Most of these studies have used aminopyrine (¹³C- or ¹⁴C-labelled), as a probe that becomes oxidised to CO₂ during substrate metabolism The CO₂ thus formed ultimately appears in exhaled breath which is easily sampled. If it is known which carbon atom in the skeleton of the substrate is oxidised to CO₂, then a position specific ¹³C-labelled substrate can be used as a probe for a particular physiological process. After administration of the ¹³C-labelled probe, breath samples are taken at known intervals. These samples are measured by isotope ratio mass spectrometer (IRMS) to detect the presence and amount of DCO₂ in the breath. IRMS systems are capable of highly precise measurement of small isotopic enrichment down to natural abundance level. Moreover, stable isotope labelled compounds offer a risk-free alternative to radioisotopes and radioisotope labelled compounds, and the risk of uncertain long term side-effects associated with radioactive tracers is completely eliminated. There are also further cost benefits such as the avoidance of the indirect costs associated with storage and handling radioactive isotopes which also reduces safety hazards for staff. The test can be made simple enough so that administration of the stable isotope tracer and collection of breath or urine samples may be carried by a technician or a nurse. This enables the non-specialist (e.g. GP) to make use of this technique as no specialist qualification (such as that of a consultant) or special equipment is required for the test procedure-Doe to its non-invasive and uncomplicated nature, the ¹³CO₂-breath test can be carried out almost anywhere as it requires neither labour intensive procedures nor special equipment. It thus consumes only a fraction of the costs of endoscopies, biopsies, metabolic ratio tests from blood samples, histology, and bacterial cultures. Taking all its advantages into account, the ¹³CO₂-breath test provides a very attractive alternative to traditional invasive methods, especially when repeat testing or monitoring is required which are difficult using most current methods and often impossible as they would involve repeated exposure to ionising radiation.

However, in the course of the abovementioned studies using ¹³C- or ¹⁴C-labelled aminopyrine as probe, a number of observations were wade. (1) Due to genetic polymorphism(s), healthy volunteers showed already a wide inter-individual variability that could be grouped into fast, tedium and slow metabolizers based on cytochrome P450 enzyme systems (CYP450). (2) CYP450 enzyme activity can be induced or inhibited by pharmaceutical drugs thus giving rise to false-negatives or false-positives, respectively. (3) The low selectivity of the aminopyrine breath test (ABT) is also thought to be caused by the fact that aminopyrine can be oxidised by two P450 enzyme systems, CYP2B6 and CYP3A4{Quantz, Sellke, et al. 1993 257/id}{Rating & Langhans 19917 258/id}.

In initial experiments by the present inventors [¹³C-carboxyl]-L-phenylalanine was used as a potential probe to assess liver function. However, this test suffered from Insufficient specificity because of the impaired enzyme activity found in PXU heterozygotes. The only way to eliminate false-positives results, would have been to increase the range of normal values which would have reduced the discrimination index of the test.

It is an object of the present invention to obviate and/or mitigate at least one of the aforementioned disadvantages.

Broadly speaking, the present invention provides a novel liver function breath test, which may reduce the problems imposed by inter- and/or intra-individual variability encountered in prior-art liver breath tests. The present invention in one embodiment is based on a two-tier approach in which a first test (A) serves as internal reference point for a subsequent test (B).

In a first aspect the present invention provides a method for testing liver function in a subject, the method comprising the steps of;

a) providing a dose of a labelled substrate (A), at the start of a first time interval, to the subject, wherein the substrate (A) is capable of being metabolized by the liver of the subject to generate labelled CO₂ which is detectable in exhaled breath of the subject;

b) obtaining samples of exhaled breath from the subject at separate time points during the first time interval and determining a level of labelled co in each breath sample such that a maximum level of labelled Co, generation is determined;

c) providing a further dose of the labelled substrate (A) and a substrate (B) which is substantially incapable of being metabolised by the liver of the subject to generate CO., at the start of a second time interval, wherein the labelled substrate (A) and substrate (B) are, capable of reacting so as to enable substrate (B) to be excreted;

d) obtaining samples of exhaled breath from the subject at separate time points during the second time interval and determining a level of labelled CO₂ generated from unreacted labelled substrate (A) in each breath sample such that a maximum level of labelled CO₂ generation is determined; and

e) assessing liver function by determining the ratio of the maximum level of labelled CO₂ generated during the second time interval to the maximum level of labelled CO₂ generated during the first time interval.

It is understood that according to the present invention a step to equilibrate the subject's body pool of substrate (A) can be encompassed prior to step a) of the method for testing liver function in a subject according to the present invention as described above. For example, this would entail providing an initial dose of unlabelled substrate (A), prior to the start of the first time interval, to the subject for equilibration of the subject's body pool of substrate (A).

Thus, in a further embodiment the present invention provides a method for testing liver function in a subject, the method comprising the steps of;

a) providing an initial dose of an unlabelled substrate (A), before the start of the first time interval, to the subject for equilibration of the subject's body pool of substrate (A);

b) providing a dose of a labelled substrate (A), at the start of a first time interval, to the subject, wherein the substrate (A) is capable of being metabolised by the liver of the subject to generate labelled CO₂ which is detectable in exhaled breath of the subject;

c) obtaining samples of exhaled breath from the subject at separate time points during the first time interval and determining a level of labelled CO₂ in each breath sample such that a maximum level of labelled CO₂ generation is determined;

d) providing a further dose of the labelled substrate (A) and a substrate (S) which is substantially incapable of being metabolised by the liver of the subject to generate CO₂, at the start of a second time interval, wherein the labelled substrate (A) and substrate [B) are capable of reacting so as to enable substrate (B) to be excreted;

e) obtaining samples of exhaled breath from the subject at separate time points during the second time interval and determining a level of labelled CO₂ generated from unreacted labelled substrate (A) in each breath sample such that a maximum level of labelled CO₂ generation is determined; and

f) assessing liver function by determining the ratio of the maximum level of labelled CO₂ generated during the second time interval to the maximum level of labelled CO₂ generated during the first time interval.

Typically, providing a dose of substrate (A) or (B) to the subject according to the present invention is achieved, for example by oral administration. Alternatively, substrate (A) or (B) may be provided to the subject in a suitable form via for example injection into the bloodstream. Preferably, substrate (A) or (B) is provided to the subject by oral administration as a solid food product for example, a flapjack or biscuit/cookie as described for example in patent application no GB0103097.2 (completed as PCT/GB02/00528).

Alternatively, substrate (A) and/or (B) may be provided to the subject as a liquid meal.

According to the present invention the term “dose” refers to a level of substrate (A) provided to the subject required to be metabolised by the subject's liver and produce a level of labelled CO₂ detectable in exhaled breath. The level of dose provided to the subject is dependent on the subject's weights For example, 150 mg of substrate (A) for a subject weight of 44.5 to 95.2 kg,

It is to be appreciated that the to subjects according to the present invention relates in general to human subjects. However, the present invention may also be conducted on animals such as horses, cows, sheep, dogs, cats and the like. The skilled reader would naturally understand that the size of “dose” provided to the subject would require to be varied according to the size of animal being tested. Thus, for example, it may be expected that the size of dose used to test the liver function of a horse may be 2-4 times larger than that described above for human testing. Moreover, the dose may be a third to half the size when used to test cats and dogs for example.

As mentioned above substrate (A) according to the present invention is a substrate that can be metabolised in the liver to generate CO₂ which is detectable in the breath of a subject. Preferably, said substrate (A) is an amino acid which may be metabolised in the liver mitochondria. Particularly, said substrate (A) is oxidised to CO₂ during substrate metabolism with the CO₂ thus formed appearing ultimately in exhaled breath which is easily sampled and measured. Most preferably, said substrate/amino acid is glycine or glucuronic acid.

Advantageously, substrate (A) is labs lied with a non-radioactive label that can be measured without risk in exhaled breath when substrate (A) is oxidised to labelled CO₂ during metabolism by the liver. Preferably, the label is a stable isotope for example, ¹³C or ¹⁴C. Optionally, if it is known which carbon atom in the skeleton of substrate (A) is oxidised to CO₂, then a position-specific ¹³C- or ¹⁴C-labelled substrate (A) can be used as a probe for a particular physiological process.

Substrate (B) according to the present invention is a substrate that is substantially incapable of being metabolized by the liver of the subject to generate CO₂. Typically, substrate (B) may only be excreted from the body by reacting with substrate (A), for example, via conjugation with substrate (A). Preferably, substrate (E) is a foreign substance or xenobiotic, which is not harmful to the body. Most preferably, substrate (B) is benzoic acid or sodium benzoate, a widely used and approved food preservative. Typically, benzoic acid is not broken down by the body (ie. the liver) and is disposed of by conjugating it with an equimolar amount of substrate (A), for example glycine to make hippuric acid, a water soluble compound that is excreted in the urine via the kidneys.

Determining a maximum level of labelled CO₂ in exhaled breath according to the present invention is generally achieved using for example stable isotope ratio mass spectrometer (IRMS) systems, capable of highly precise measurement of small isotopic enrichment down to natural abundance level. Thus, preferably, the label according to the present invention is a stable isotope, for example ¹³C or ¹⁴C, which may be measured using IRMS in exhaled breath samples. Determining ¹³CO₂ in ¹³CO₂-breath tests are documented further in patent application GB0103097.2 (completed as PCT/GB02/00528). Specific details of acts breath tests may be found in, for example, Schommartz et al. In summary however, a test patient provides an initial breath sample in order to establish a natural background or base line of ¹³CO₂. The patient is then provided with for example ¹³C-labelled substrate (A) and breath samrples are taken over a time course and the amount of ¹³CO₂ at each sample determined for assessment of liver function.

Breath samples are thus measured for ¹³CO₂ content above base line, for example, the mean of 3 pre-test samples. The time to peak of ¹³CO₂ exhalation, ¹³CO₂ exhalation in percentage of given dose [% dose/hour] (percentage dose recovery (PDR)) and cumulative ¹³CO₂ exhalation over time may be determined. The “maximum level” of labelled CO₂ according to the present invention is the peak level ¹³CO₂ exhalation or ¹³CO₂ exhalation in percentage of given dose [% dose/hour] (percentage dose recovery (PDRₘₐₓ)) as described above. This “maximum level” is “determined” for example by plotting values of ¹³CO₂ levels measured in breath samples using IRMS, for example, over a time course on a graph and reading the “maximum level of labelled CO₂” from the graph. Generally speaking, plotting of the labelled CO₂ level points and reading of the “maximum level of labelled CO₂” is carried out manually. Preferably, both these steps and ultimately the calculation of the “maximum level of labelled CO₂” may be determined by computer using appropriate software.

A preferred method or testing liver function adopts a two test principle for example test (A) and test (B), and relating the results of these two tests For example, “¹³C-labelled glycine (substrate (A)) is used as a probe for test (A) and again for subsequent test (B). In test (B) however, ¹³C-glycine is provided with at equimolar amount of sodium benzoate (substrate (B)) for example. The latter substrate cannot be metabolised by the human or animal body and has to be excreted via reaction or conjugation with glycine The ability to conjugate, for example sodium benzoate with glycine is regarded to be a good indicator of liver function.

Advantageously, by relating the results of test (A) and test (B) the co-efficient of variation of the method for testing liver function will be reduced. This eliminates both inter- and intra-individual viability thus improving both selectivity and specificity. By looking at the ratio of PDRₘₐₓ instead of an absolute measured value, parameters subject to individual physiology such as endogenous carbon dioxide production will cancel each other out as they will be part of both numerator and denominator.

In a healthy patient, during test (A) ¹³C-glycine for example will be predominantly metabolised to ¹³CO₂ giving rise to a substantial ¹³CO₂ signal in breath samples whereas in test (B) ¹³C-glycine will be conjugated to the simultaneously administered sodium benzoate. Since this reaction will strongly compete with the breakdown of ¹³C-glycine, the ¹³CO₂ exhalation will be reduced Levels of ¹³CO₂ are measured as a maximum percentage dose recovery (PDRₘₐₓ) of CO₂ in exhaled breath samples taken at regular intervals over a known period of time for test A (PDRₘₐₓ(A)) and test a (PDRₘₐₓ(B)). Thus, in a healthy patient the ratio of PDRₘₐₓ(B) over PDRₘₐₓ(A) will be small (eg. 0.6 and less). In a patient suffering from chronic liver disease, the rate of glycine conjugation will be less than in a healthy patient, do there will be less competition with glycine breakdown resulting in a higher ¹³CO₂ signal for test B compared to that or a healthy patient. One would therefore expect to observe a PDRₘₐₓ(B)/PDRₘₐₓ(A) ratio between 0.7 and 1.0 depending on the degree of severity of disease A PDRₘₐₓ(B)/PDRₘₐₓ(A) ratio of 1.0 would indicate severely impaired liver function.

A further aspect of the present invention is based on the inventor's observations that the time to peak of ¹³CO₂ exhalation or ¹³CO₂ exhalation in percentage of given dose [% dose/hour] i.e. PDFₘₐₓ was significantly delayed in patients with known liver function deficiencies for example, Hepatitis C compared with normal control subjects.

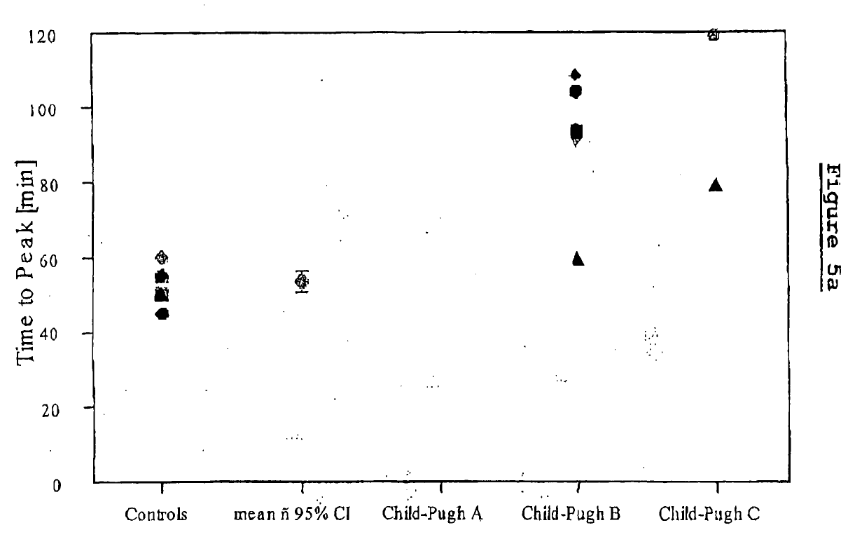

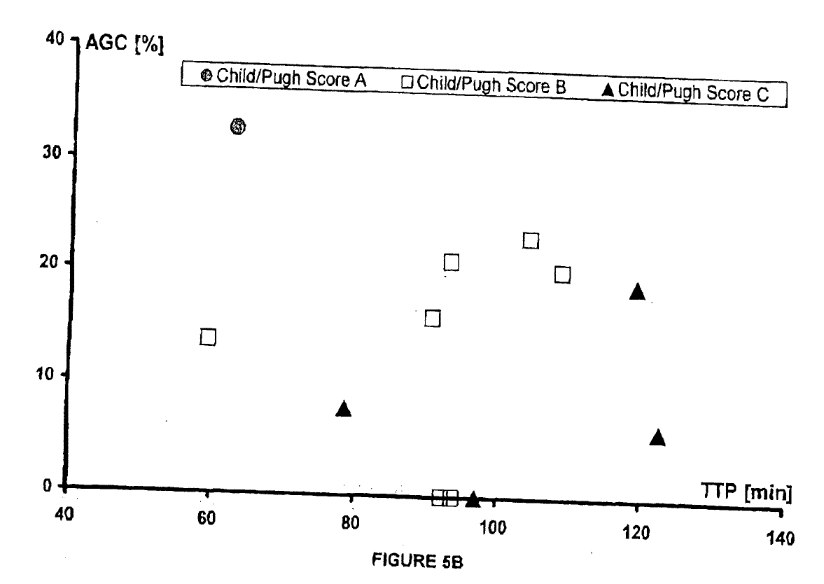

For example, in FIG. 5 the delay in the time to peak at ¹³CO₂ exhalation or ¹³CO₂ exhalation in percentage of given dose [dose/hour] i.e, PDRₘₐₓ, in discussed patients may be indicative of reduced blood flow through the liver due to for example, portal hypertension.

Thus, the present invention also provides methods for testing liver function based on observing the difference between the apparent percentage level of reaction or conjugation between substrate (A) and substrate (B) in a control versus a test subject.

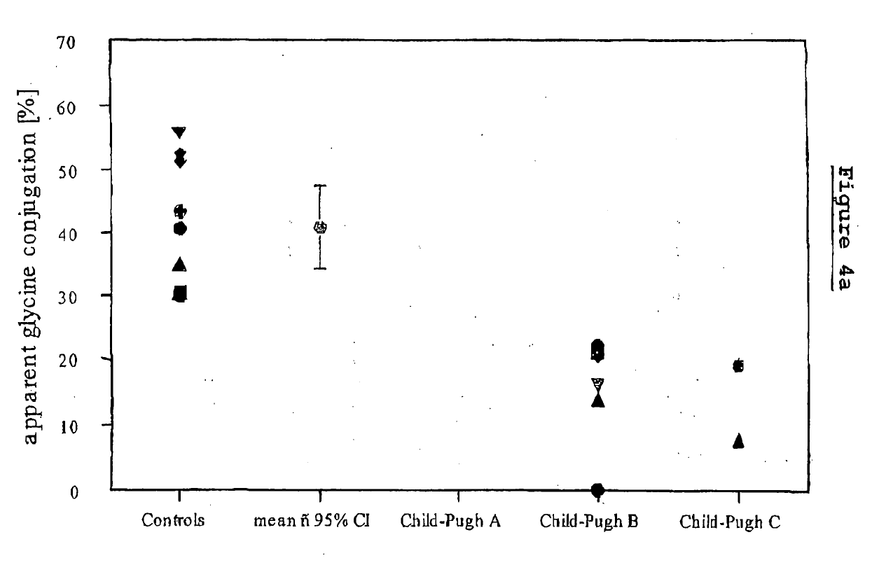

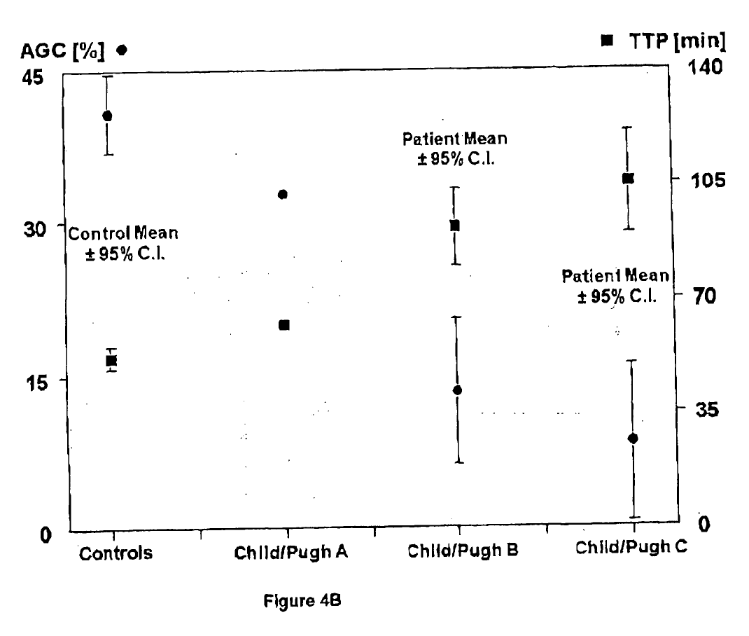

For example, FIG. 4 shows that in subjects with know liver function deficiency (i.e. Hepatitis C) that apparent glycine conjugation is decreased with respect to control. It is understood that “control” according to the present invention relates to subjects with substantially normal liver function.

Applications of the present invention include:

(i) diagnosis and prediction of outcome of liver dysfunction;

(ii) non-invasive monitoring of disease status on a regular basis (especially in children);

(ii) detection of changes in liver function due to adverse drug reactions (ADRS) or chronic medication, and

(iv) prediction of phenotypes with impaired drug metabolising capability.

In a further aspect of the present, invention there is provided a kit for testing liver function in a subject wherein the kit comprises:

a) a first product comprising a labelled substrate (A), wherein the substrate (A) is capable of being metabolised by the liver of the subject to generate labelled CO₂, which is detectable in exhaled breath of the subject; and

b) a second product comprising a substrate (B) or a labelled substrate (A) and substrate (B), wherein substrate (B) is substantially incapable of being metabolised by the liver of the subject to generate CO₂ and wherein the labelled substrate (A) and substrate (B) are capable of reacting so as to enable substrate (B) to be excreted.

Typically, providing a dose of substrate (A) or (B) to the subject according to the present invention is achieved, for example by oral administration. Alternatively, substrate (A) and/or (B) may be provided to the subject in a suitable form via for example injection into the bloodstream. Preferably, substrate (A) and/or (B) is provided to the subject by oral administration as a solid food product for example, a flapjack or biscuit/cookie as described for example in patent application no. GB0103097.2 (completed as PCT/GB02/00528). Alternatively, substrate (A) and/or (B) may be provided to the subject as a liquid meal.

The invention will now be illustrated by way of the following figures and Examples; wherein

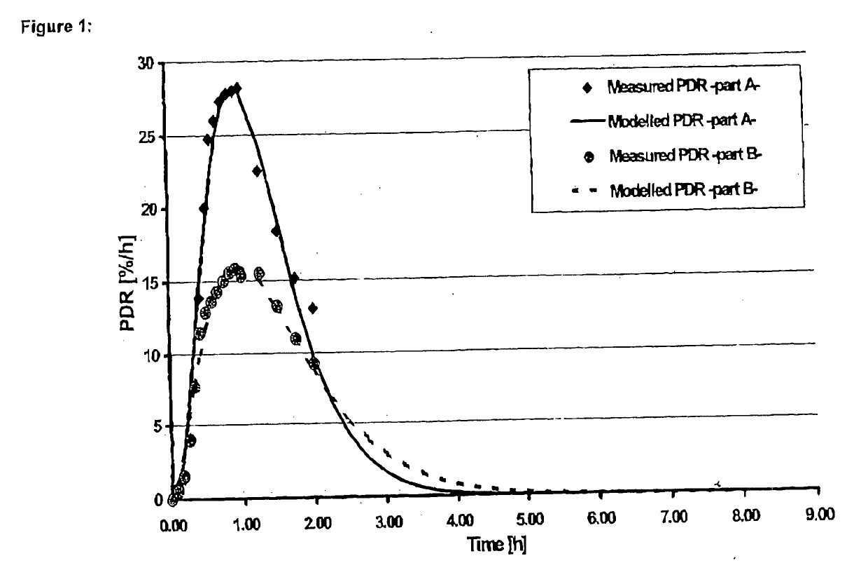

FIG. 1—Breath test of control subject L. Part A and part B were carried out on 2 consecutive days. B/A ratio=0.57.

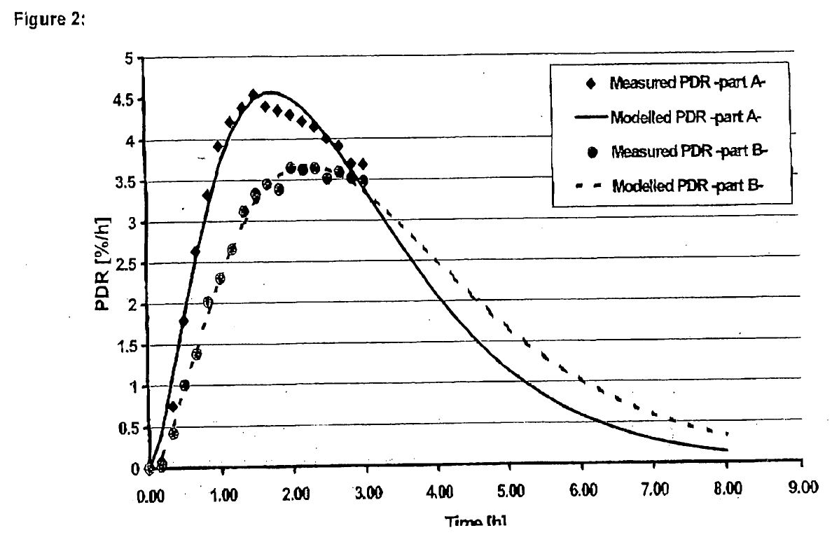

FIG. 2—Breath test on patient NELP002. Part A and part B were caried out on 2 consecutive days. B/A ratio 0.86.

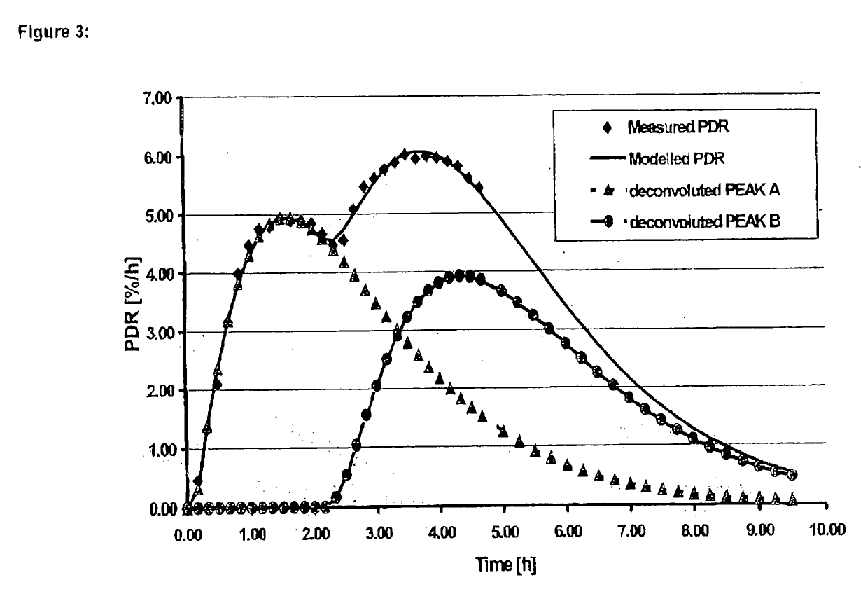

FIG. 3—Breath test on patient NHLP012. “One day” protocol combining part A and part B into a 4.5 h test. B/A ratio=0.80.

FIG. 4A—Apparent ¹³C-glycine conjugation calculated from peak PDRₙ ratios for controls and patients.

FIG. 4B—Further results of apparent ¹³C-glycine conjugation calculated from peak PDRₙ ratios for controls and patients. Results from two-phase breath test measuring glycine conjugation with benzoate show a clear distinction between patients and controls. AGC=apparent glycine conjugation; TTP=time to peak of recovered dose.

FIG. 5A—Time to peak maximum of ¹³C-glycine PDRₙ or controls and patients.

FIG. 5B—Further results of time to peak maximum of ¹³C-glycine PDRₙ for controls and patients. The individual patient time to PDR peak valves (TTP) are plotted versus their corresponding AGCS. The absence of any correlation between TTP and AGC indicates that observed AGCs are not due to artefacts induced by e.g. limited substrate delivery to the liver caused by shunting or poor blood flow.

### EXAMPLE 1

### Liver Function Breath Test

Variation 1

The test is carried out in the morning on an empty stomach after an overnight fast.

To equilibrate the body's glycine pool, an oral bolus of 3 g of glycine dissolved in 200 ml of water is given 1 hour prior to tracer administration.

At t=0 min, a weight adjusted dose of [¹³C−1]-glycine (eg. 150 mg for 44.5 to 95.2 kg body weight) was given orally. Breath samples are taken subsequently every 10 minutes over a period of 300 minutes.

At t=160 min, a second dose of [¹³C−1]-glycine was given, followed by three equal dozes of sodium benzoate (eg. 284.2 mg) at 170, 180 and 190 minutes.

Breath samples are measured for ¹³CO₂ content above base line, ie. the mean of 3 pre-test samples. Time to peak of ¹³CO₂ exhalation, ¹³CO₂ exhalation in percentage of given dose [% dose/hour] and cumulative ¹³CO₂ exhalation over time are determined. Mathematical modelling of the various curves is used to de-convolute the dose recovery curves of parts A and B.

Variation 2

The breath test consisted of two parts, A and B, that were at first carried out on 2 consecutive days and later combined into an one-day protocol.

Part A; After an overnight fast, subjects were given orally a weight adjusted dose of the tracer ¹³C-glycine dissolved in water

Part B: After an, overnight fast, subjects were given orally the same amount of tracer plus an optimised amount of sodium benzoate.

For the one-day protocol, part B commenced 130 min after the start of part A.

Breath samples were collected in 10 min intervals.

¹³C-abundance in exhaled CO₂ was measured by isotope ratio mass spectrometry (IRMS) and from these data percentage dose recovery (PDR [%/h]) and cumulative PDR (cPDR) were calculated and mathematically modelled.

10 volunteers (5 women, 5 men) with no medical history of liver problems were used as control group.

9 in-patients (6 women, 3 men) with varying stages of liver failure and different disease etiology were tested. Ethical approval for this ¹³CO₂-breath test, study was sought and obtained from the Tayside Healthboard Ethics Committee.

TABLE 1
Average ¹³C-Glycine Conjugation and Time to
Peak PDRₘ for Controls and Patients

| | | S.D | | S.D |
|---|---|---|---|---|
| | Controls | (n = 11) | Patients | (n = 9) |
| Apparent glycine | 40.83 | ±6.57 | 9.35 | 17.11 |
| conjugation[%] | | | | |
| Time to peak PDR [min] | 53.50 | ±5.30 | 94.98 | 17.14 |

Results

Apparent glycine conjugation (GCₐ) was calculated from [1−((PDRₘ{B}/PDRₘ{A}×100)], using modelled PDRₙ values for parts A and B at peak for the control group, mean PDRₘ ratio was 0.59 and GCₐ. Was hence calculated to be 40.83%. Its co-efficient of variation was 16.1%.

The highest GCₐ-value in patients observed so far was 23.26.% for a patient suffering from Hepatitus C (Child class B, Pugh score 7).

Time to peak PDR in patients was significantly delayed compared to the control group. We hypothesised that this might be indicative of reduced blood flow through the liver due to eg. portal hypertension.

A further example, results of which are depicted in FIG. 4B, shows that a two-phase breath test probing hepatic glycine conjugation can distinguish controls from patients with a Child-Pugh score of B, yielding an apparent glycine conjugation of 40.8±3.9% and 13.5±7.2% (mean±95% confidence limits), respectively.

### REFERENCES

Schommartz, B., D. Ziegler and P Schadewaldt. Significance of diagnostic parameters in [¹³C]-octanoic acid gastric-emptying breath tests. (1998). Isotopes Environ. Health Stud., 34:135-143.

1. A method for testing liver function in a subject, the method comprising the steps of:
a) providing a dose of a labelled substrate (A), at the start of a first time interval, to the subject, wherein the substrate (A) is capable of being metabolised by the liver of the subject to generate labelled CO₂ which is detectable in exhaled breath of the subject;
b) obtaining samples of exhaled breath from the subject at separate time points during the first time interval and determining a level of labelled CO₂ in each breath sample such that a maximum level of labelled CO₂ generation is determined;
c) providing a further dose of the labelled substrate (A) and a substrate (B) which is substantially incapable of being metabolised by the liver of the subject to generate CO₂, at the start of a second time interval, wherein the labelled substrate (A) and substrate (B) are capable of reacting so as to enable substrate (B) to be excreted;
d) obtaining samples of exhaled breath from the subject at separate time points during the second time interval and determining a level of labelled CO, generated from unreacted labelled substrate (A) in each breath sample such that a maximum level of labelled CO₂ generation is determined; and
e) assessing liver function by determining the ratio of the maximum level of labelled CO₂ generated during the second time interval to the maximum level of labelled CO₂ generated during the first time interval.

2. The method according to claim 1, the method further comprising the step of providing an initial dose of an unlabelled substrate (A), prior to step a), to the subject for equilibration of the subject's body pool of substrate (A).

3. The method according to either of claims 1 or 2 wherein said dose of substrate (A) or (B) is provided to the subject by oral administration or by injection into the bloodstream.

4. The method according to any preceding claim wherein the dose of substrate (A) provided to the subject is at a level required to be metabolized by the subject's liver to produce a level of labelled CO₂ detectable in exhaled breath.

5. The method according to any preceding claim wherein said dose is dependent on the subject's Weight.

6. The method according to any preceding claim wherein the subject is human or animal.

7. The method according to any preceding claim wherein substrate (A) is an amino acid which is metabolised in the liver mitochondria.

8. The method according to any preceding claim wherein said substrate/amino acid is glycine or glucuronic acid.

9. The method according to any preceding claim wherein substrate (A) is labelled with a non-radioactive label.

10. The method according to claim 9 wherein the label is a stable isotope such as ¹³C or ¹⁴C.

11. The method according to any preceding claim wherein substrate (B) is only excreted from the body on reaction with substrate (A).

12. The method according to any preceding claim wherein substrate (B) is benzoic acid or sodium benzoate.

13. The method according to any preceding claim wherein determination of said level of labelled CO₂ in exhaled breath is achieved using stable isotope ratio mass spectrometer (IRMS) systems.

14. The method according to any preceding claim wherein the ratio of levels detected in steps b) and d) is 0.6 or less in a healthy subject.

15. The method according to claims 1-13 wherein the ratio of levels detected in steps b) and d) is between 0.7 and 1.0 in a patient suffering from chronic liver disease.

16. The method according to any preceding claim wherein a significantly delayed time to reach a maximum level of labelled CO₂ exhalation or labelled CO₂ exhalation in percentage of given dose in a subject is indicative of a deficiency in liver function compared with normal control subjects.

17. The method according to any preceding claim to test liver function wherein the apparent percentage level of reaction or conjugation between substrate (A) and substrate (B) in a control subject is compared with that of a test subject.

18. The method according to claim 17 wherein conjugation is decreased in subjects with known liver function deficiency compared to control subject.

19. A kit for testing liver function in a subject wherein the kit comprises:
a) a first product comprising a labelled substrate (A), wherein the substrate (A) is capable of being metabolised by the liver of the subject to generate labelled CO₂, which is detectable in exhaled breath of the subject; and
b) a second product comprising a substrate (B) or a labelled substrate (A) and substrate (B), wherein substrate (B) is substantially incapable of being metabolised by the liver of the subject to generate CO₂ and wherein the labelled substrate (A) and substrate (B) are capable of reacting so as to enable substrate (B) to be excreted.

20. The kit according to claim 19 wherein the first and/or second products are provided to the subject in the form of a solid food product.
